(19) United States
(12) Patent Application Publication
(10) Pub. No.: US 2005/0245914 A1
(43) Pub. Date: Nov. 3, 2005

(54) Vein-removing device on a tape

(76) Inventors: Michel Camus, Templemars (FR); Assem Mikati, Avesnes Sur Helpe (FR)

Correspondence Address: WENDEROTH, LIND & PONACK, L.L.P., 2033 K STREET N. W., SUITE 800, WASHINGTON, DC, 20006-1021, US

(21) Appl. No.: 11/106,527

(22) Filed: Apr. 15, 2005

(30) Foreign Application Priority Data
Apr. 16, 2004 (FR) 0404025

(51) Int. Cl. A61B 17/00

(52) U.S. Cl. 606/1

(57) ABSTRACT

The invention relates to a vein-removing device, or stripper, which uses an absorbent and/or coagulative tape for reducing the risk of haematomas. The vein-removing device comprises a wire or cable for extracting the vein, which wire or cable is connected at its end to a tape of natural or synthetic, blood-absorbing, gripping material, which tape is of the requisite length for vein-removal and which supports and grips the vein for the progressive invagination thereof over all or part of its length, as the vein-removing process progresses.

The degradation of the saphenous veins of the legs and the pain which those veins cause have led the medical profession to carry out total or partial removal of the saphenous veins in order to eliminate either the compression of the saphenous nerve which adjoins the veins, and which is a source of pain, or the unsightly appearance of varices which are ampullae resembling a hernia, from one or more sites on the vein concerned.

This operation called vein-removal or stripping, which is generally performed by surgeons, has been carried out for a very long time and the equipment which has enabled this surgical act to be performed has evolved over time.

The first strippers were composed of an olive of more or less large size which was secured to a cable and which was introduced into the leg at the upper or lower portion of the vein concerned.

Traction on the cable pulled the ball thus secured and “literally tore” the vein from its location.

This method of intervention which is unfortunately still practiced owing to its low cost is, however, on the decline in favour of stripping by invagination.

This method consists in folding the vein in on itself and therefore in causing far less traumatism and bleeding and a marked decrease in the risk of paraesthesia or excessive terminal sensitization of the foot than does the initial method.

However, a pathological vein, which is delicate, or a tendency of the vein to break during traction, then requires, during the same operation, the use of one of the olives which accompany this type of stripper, and therefore constrains the practitioner to terminate the stripping in the above-mentioned manner. (cf Prof Chevalier, Prof Becquemin, Dr Valici etc. . . . )

The risks run are lesser because a portion of the vein has already been extracted by this method and it is therefore only the remainder of the vein which is still in place, and therefore less voluminous, which merely has to be withdrawn.

We therefore propose another alternative to this type of stripping while preserving the principle of invagination thereof.

The invention will be better understood on reading the following description which is given by way of example and with reference to the drawings in which:

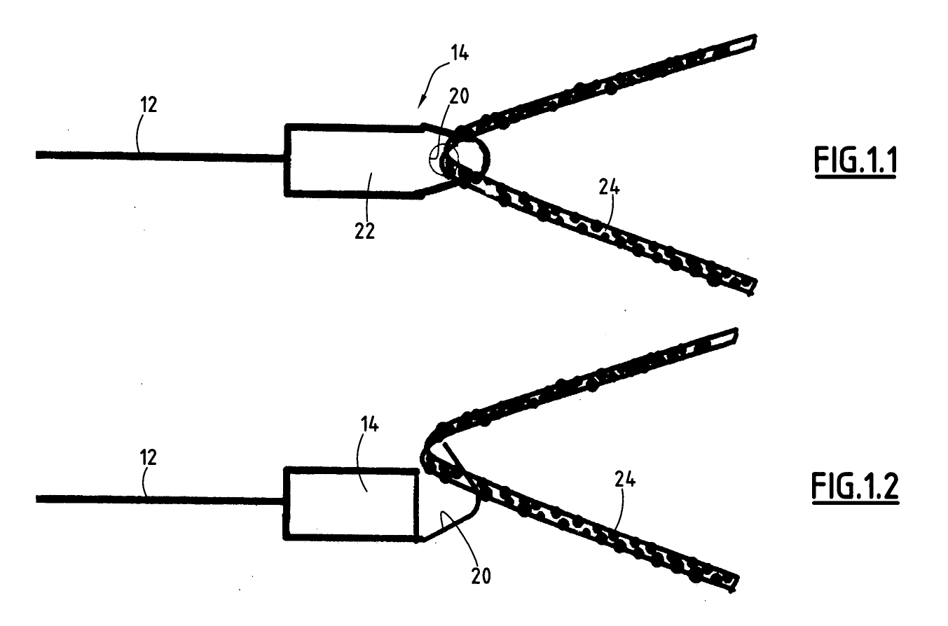

FIG. 1.1 is a partial elevation of a surgical vein-removing instrument according to the invention;

FIG. 1.2 is a view identical to that of FIG. 1 of a variant of a surgical vein-removing instrument;

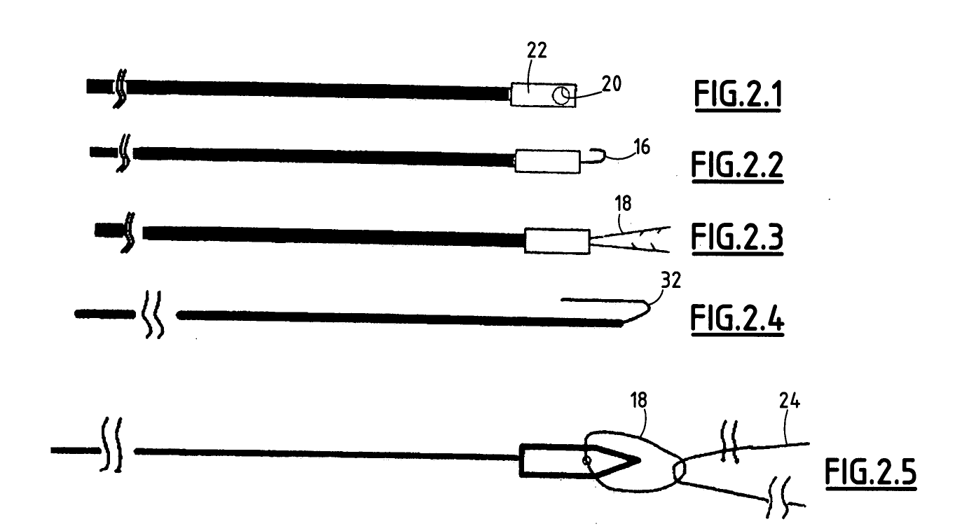

FIGS. 2.1, 2.2, 2.3, 2.4 and 2.5 are elevations of other variants; and

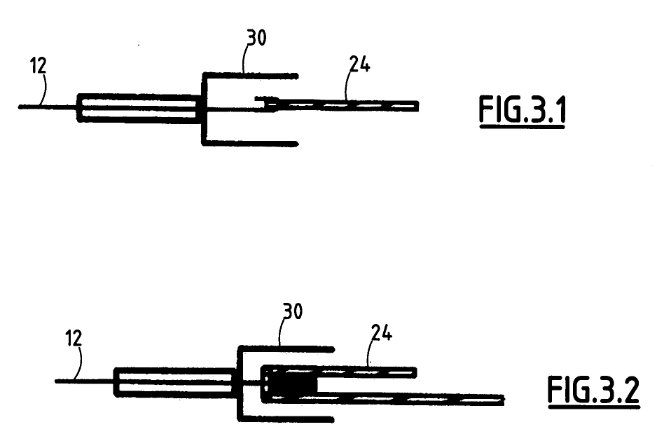

FIGS. 3.1 and 3.2 are yet other variants.

Thus the surgical vein-removing instrument or stripper may be composed of a cable 12, or traction wire, which is a device still used in the above-mentioned equipment.

The cable or wire is provided at its end with a device 14, such as a hook 16 (FIG. 2.2) or gripping pincers (FIG. 2.3) or a pivoting ring 18 (FIG. 2.5) or any other device, such as an adequate opening 20, in an end-piece 22 (FIGS. 1.1 and 2.1), enabling a tape 24 of crepe or a tape of gauze, which is sufficiently long to extend well beyond the length of the member concerned and, more precisely, the length of the vein to be extracted, to be secured, or threaded through within the extent thereof. The length of the tape 24 is to be understood as the length of the folded tape, that is to say, the distance separating the folded portion of the tape from its furthermost free end if the two portions have different lengths. In practice, the two portions advantageously have the same length equal to the length of the tape.

The tape is advantageously composed of crepe rather than gauze, crepe being a fabric having a characteristic undulating appearance which is obtained by using highly twisted threads called “crepe threads ”.

Stripping is then carried out on the tape and no longer on a wire or on a tube.

The quality of this tape and the thickness which it represents are necessary and sufficient for the vein to form one piece therewith and to be extracted by being turned inside out as described above.

The tape in question is advantageously of requisite and sufficient length for, when all of the vein has come out, the remainder of the tape still to be in place in the leg, and remains there for a period of time, for example, the time necessary for the associated phlebectomy, and its capillarity is exploited to absorb the blood directly in situ and also to reduce the still-present risk of post-operative haematomas. The tape will therefore be of an absorbent nature.

The surgical vein-removing instrument comprises a wire or cable for extracting the vein, which wire or cable is connected at its end to a tape 24 of natural or synthetic, gripping, (FIG. 1.3) blood-absorbing material, which tape is of the requisite length for vein-removal and which supports and grips the vein in the course of the progressive invagination thereof over all or part of its length, as the vein-removing process progresses. It is even possible to conceive of a tape impregnated with a coagulation agent which, while the remaining element of the tape stays in situ, would therefore facilitate the coagulation of the surrounding tissue and the breakage sites of the collaterals left in position, which are the source of sometimes major per- and post-operative bleeding.

The length of the tape of the instrument is greater than the length of the vein in order, once the vein has been extracted, to enable the portion of the tape located and remaining in the region concerned to absorb the blood. Advantageously, the length of the tape is at least twice as great as the length of the vein to be extracted.

All or part of the tape of the instrument comprises at least one agent promoting the coagulation of the blood in situ.

The instrument comprises a connecting device between the wire and the tape, such as a ring pivoting on the wire, or an opening 20 which is workable (FIG. 1.2) or non-workable (FIGS. 1.1 and 2.1) and which is formed in the end-piece, the tape being inserted into the ring or the opening by folding.

The instrument's tape of absorbent material is a non-smooth textile material of the “crepe” type because its unevenness then promotes the adhesion of the vein to the tape.

Moreover, the necessity of having an olive as a “safety net” in case the vein breaks is found to be no longer necessary because the vein, over the entire path of the tape, and owing to the stretching thereof, requires only adhesion to the gripping fabric and therefore a reduction in the initial tractive force represented by the mere ligature of the vein to the traction wire. On the other hand, a simple knot formed, for example, in the lower portion of the tape will perform the same function while being less traumatizing because it is less rigid. The practitioner has then only to withdraw this tape which has remained in place, when the operation is complete, in order to obtain a vein track in the leg without an excess of blood and therefore without a haematoma, which is a medical risk and which is still unsightly.

The device for securing the wire or cable and the tape to each other is covered with an element 30 (FIGS. 3.1, 3.2) suitable for facilitating the introduction of the device into the vein concerned; the wire or cable used, marked 32 in FIG. 2.4, is deformable with pincers to form an abutment for the sliding tube surrounding it.

It will be appreciated that, with such an instrument, the vein which has been turned inside out is supported internally on the tape which, owing to its structure, matches the shape of the vein and reinforces it, thus avoiding accidental breakage.

1. Surgical vein-removing instrument, characterized in that it comprises a wire or cable (12; 32) for extracting the vein, which wire or cable is connected at its end to a tape (24) of natural or synthetic, blood-absorbing, gripping material, which tape is of the requisite length for vein-removal and which supports and grips the vein for the progressive invagination thereof over all or part of its length, as the vein-removing process progresses.

2. Instrument according to claim 1, characterized in that the length of the tape (24) is greater than the length of the vein in order, once the vein has been extracted, to enable the portion of the tape located and remaining in the region concerned to absorb the blood.

3. Instrument according to claim 1, characterized in that all or part of the tape (24) comprises at least one agent promoting the coagulation of the blood in situ.

4. Instrument according to claim 1, characterized in that it comprises a connecting device (14) between the wire (12) and the tape (24).

5. Instrument according to claim 4, characterized in that the connecting device (14) between the wire (12) and the tape (24) is a ring (18; 20) pivoting on the wire, the tape (24) being inserted into the ring (18, 20) by folding.

6. Instrument according to claim 1, characterized in that the tape (24) of absorbent material is a non-smooth and absorbent textile material of the “crepe” type.

7. Instrument according to claim 4, characterized in that it comprises a hook (16) for securing the traction wire (12) and the tape (24) to each other.

8. Surgical vein-removing instrument according to claim 4, characterized in that the device for securing the traction wire and the tape to each other is a pair of pincers (18).

9. Surgical vein-removing instrument according to claim 4, characterized in that the device for securing the wire or cable and the tape to each other is covered with an element (30) suitable for facilitating the introduction of the device into the vein concerned.

10. Surgical vein-removing instrument according to claim 1, characterized in that the wire or cable used (32) is deformable with pincers in order to form an abutment for the sliding tube surrounding it.

11. Instrument according to claim 2, characterized in that all or part of the tape (24) comprises at least one agent promoting the coagulation of the blood in situ.

12. Instrument according to claim 2, characterized in that it comprises a connecting device (14) between the wire (12) and the tape (24).

13. Instrument according to claim 3, characterized in that it comprises a connecting device (14) between the wire (12) and the tape (24).

14. Instrument according to claim 11, characterized in that it comprises a connecting device (14) between the wire (12) and the tape (24).

15. Instrument according to claim 12, characterized in that the connecting device (14) between the wire (12) and the tape (24) is a ring (18; 20) pivoting on the wire, the tape (24) being inserted into the ring (18, 20) by folding.

16. Instrument according to claim 13, characterized in that the connecting device (14) between the wire (12) and the tape (24) is a ring (18; 20) pivoting on the wire, the tape (24) being inserted into the ring (18, 20) by folding.

17. Instrument according to claim 14, characterized in that the connecting device (14) between the wire (12) and the tape (24) is a ring (18; 20) pivoting on the wire, the tape (24) being inserted into the ring (18, 20) by folding.

18. Instrument according to claim 2, characterized in that the tape (24) of absorbent material is a non-smooth and absorbent textile material of the “crepe” type.

19. Instrument according to claim 3, characterized in that the tape (24) of absorbent material is a non-smooth and absorbent textile material of the “crepe” type.

20. Instrument according to claim 4, characterized in that the tape (24) of absorbent material is a non-smooth and absorbent textile material of the “crepe” type.
